(19) United States
(12) Patent Application Publication
(10) Pub. No.: US 2008/0147025 A1
(43) Pub. Date: Jun. 19, 2008

(54) Pleated disposable garment

(76) Inventors: Paul T. Van Gompel, Hortonville, WI (US); LuAnn M. Beckman, Neenah, WI (US); Eric John R. Gilgenbach, Winneconne, WI (US); John T. Hahn, Merrill, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/638,937

(22) Filed: Dec. 13, 2006

(51) Int. Cl. A61F 13/15; A61M 1/00

(52) U.S. Cl. 604/358; 604/317; 604/365; 604/385.24

(57) ABSTRACT

A disposable garment for wearing about the lower torso employs pleats or folds in the waist region of the garment allowing the hip region to expand. The pleats are secured in a bond zone at the waist. An absorbent assembly is attached at the waist region and is free to expand at the hip region of the garment.

### BACKGROUND OF THE INVENTION

This invention relates generally to a disposable garment for personal wear, and more particularly to such a disposable garment with improved fit and reduced leakage.

Disposable garments are known for use in the manner of underpants for children and adults, garments worn like training pants for toddlers, and garments worn like diapers for infants. Disposable absorbent garments are designed to absorb and contain bodily fluids, but otherwise have a limited period of use before their ability to perform their intended function is exhausted. In particular, such disposable absorbent garments are intended to be discarded after one or more insults instead of being laundered.

Typically, it is desirable for disposable garments to fit close to the body of the wearer for comfort and discretion. Presently available disposable garments for wear about one's waist include a substantially liquid impermeable outer cover, a liquid permeable bodyside liner in a superposed relation with the outer cover, and an absorbent structure located between the outer cover and the bodyside liner for taking in and retaining liquid body exudates. The side edges of the garment form a pair of leg openings when the disposable garment is worn and the ends of the garment together form the waist opening thereof. Elastic components such as waist elastics, leg elastics and containment flaps are commonly used to enhance the fit and inhibit leakage from the garment.

While disposable garments have been known for many years, the materials used to construct them have continuously evolved as a result of new technologies for formulating and manufacturing disposable materials. Materials may be selected for performance or to provide a cost advantage, particularly given that many manufacturers produce disposable garments and disposable absorbent garments in very large quantities. One aspect of evolution has been the development and availability of stretchable materials to replace previously non-stretchable components in order to provide improvements in the way in which the garments fit and improvements in the range of fit of the garments.

With the advent of the availability of stretchable materials to construct disposable absorbent garments, various configurations for garments incorporating stretchable materials have been described. Simultaneous with the development of stretchable materials having lower cost and/or improved properties for use in disposable garments, developments with regard to the structural features of disposable garments have also occurred.

Even though significant and numerous advancements have occurred in the materials and structural features available for the construction of disposable garments, there remain opportunities for improvement in the fit, containment, and ventilation capacity of such garments. For example, there remains a need for a disposable garment that provides improved fit on the wearer while inhibiting leakage and keeping the waste materials contained by the garment away from the skin of the wearer.

### SUMMARY OF THE INVENTION

In one aspect of the present invention is a disposable garment for wearing about the lower torso of a person. The garment has lateral side edges, and front and back longitudinal ends defining a waist opening. The garment includes an outer layer that is in opposed relationship with an inner layer. The inner layer has a pleat located at the waist opening, and the pleat and the outer layer are joined together with a permanent bond.

In another aspect of the present invention is a disposable garment having a waist opening and two leg openings for wearing about the lower torso of a person. The garment includes a chassis made from an outer layer and an inner layer. This chassis defines a longitudinal direction, a lateral direction, lateral side edges, front and back longitudinal ends and together defining the waist opening; a front waist region and a rear waist region; and a crotch region extending longitudinally between and interconnecting the front waist region and the back waist region. The outer layer is in opposed relationship with the inner layer. The inner layer includes an absorbent assembly having lateral side folds adapted to contact a body-facing surface of the absorbent assembly; and a pair of side panels positioned at the lateral side edges of the chassis and that each defines a flap portion. The absorbent assembly and each flap portion extend from the front longitudinal end to the back longitudinal end of the chassis. An elastic waist panel is located at each of the front waist region and the rear waist region, each elastic waist panel having a waist-side edge and a crotch-side edge. A “bond means” is included that functions to permanently bond together at a first bond zone: one of the elastic waist panels, the outer layer, one of the flap portions, and one of the absorbent assembly lateral side folds. The first bond zone is located adjacent the waist opening. In addition, there is a “limited bond means” that functions to permanently bond together at a second bond zone (with the exclusion of the outer layer): one of the elastic waist panels, one of the flap portions, and one of the absorbent assembly lateral side folds. The second bond zone is located directly adjacent the first bond zone, and aligned in the longitudinal direction therewith.

In yet another aspect of the present invention there is a disposable absorbent for wearing about the lower torso of a person, the garment defining a waist opening, a front waist region, and a back waist region. The garment includes an outer layer in opposed relationship with an inner layer. The inner layer and the outer layer are pleated to form one or more pleats at the front waist region or the back waist region. One or more pleats include a permanent bond at a bond zone.

The foregoing paragraphs have been provided by way of introduction, and are not intended to limit the scope of the following claims. The following embodiments will be understood by reference to the detailed description taken in conjunction with the accompanying drawings.

### BRIEF DESCRIPTION OF THE DRAWINGS

FIG. 1 is a top plan view of one embodiment of a disposable garment of the present invention in the form of a diaper illustrated in an unfolded and laid flat condition to show the surface of the diaper that contacts the skin of the wearer;

FIG. 2 is a top plan view of an embodiment of a disposable garment similar to FIG. 1 but with portions cut away to reveal internal construction, and non-tapered leg elastics;

FIG. 3 is an exploded cross-section taken in the plane of line 3-3 in FIG. 1;

FIG. 4 is a top perspective view of another embodiment of a disposable garment of the present invention in the form of a diaper illustrated in an unfolded and laid flat condition;

FIG. 5 is an exploded cross-section taken in the plane of line 5-5 of FIG. 1; and

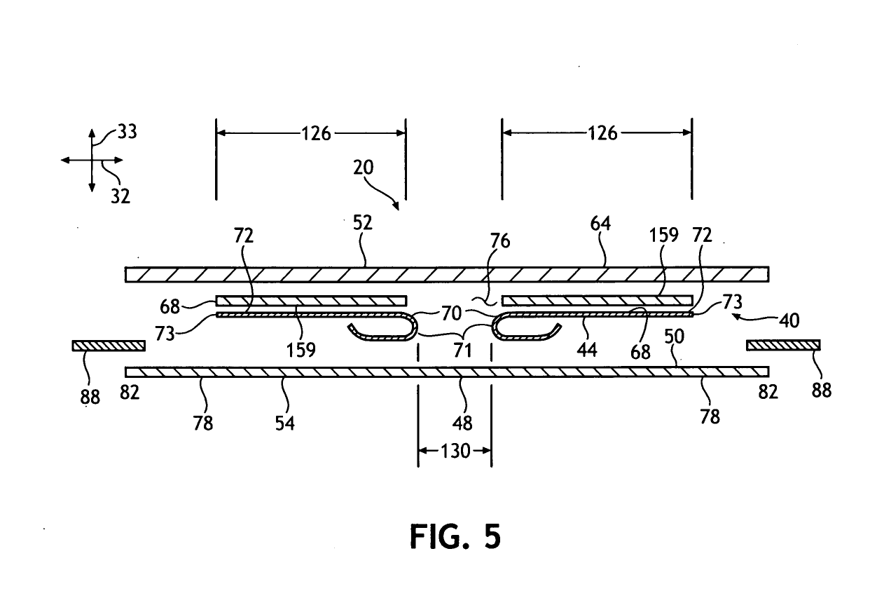

FIG. 6 is an exploded cross-section taken in the plane of line 6-6 of FIG. 1.

Corresponding reference characters indicate corresponding parts throughout the several views of the drawings.

### DETAILED DESCRIPTION OF THE INVENTION

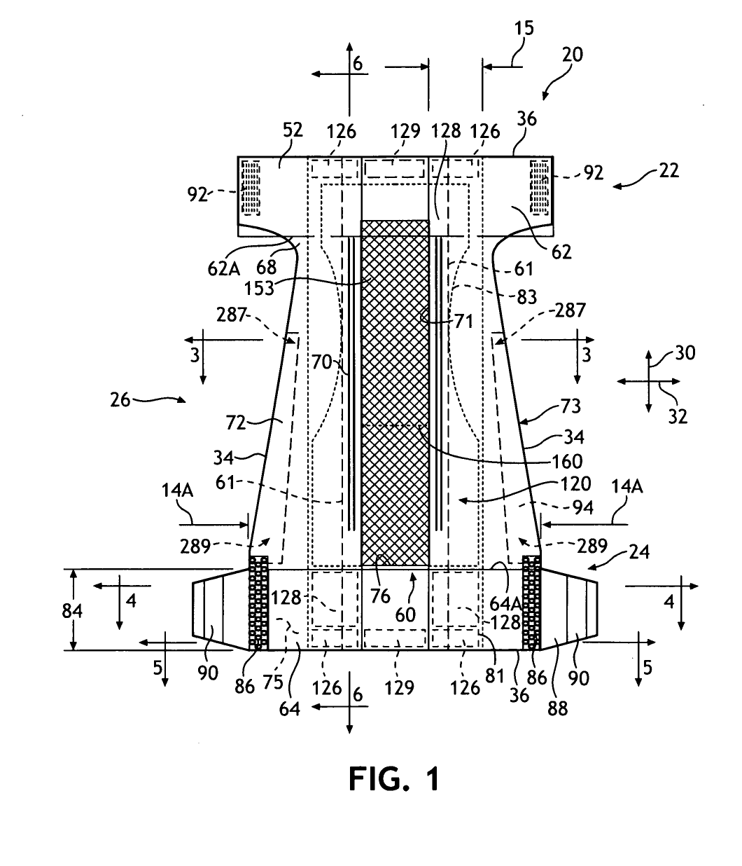

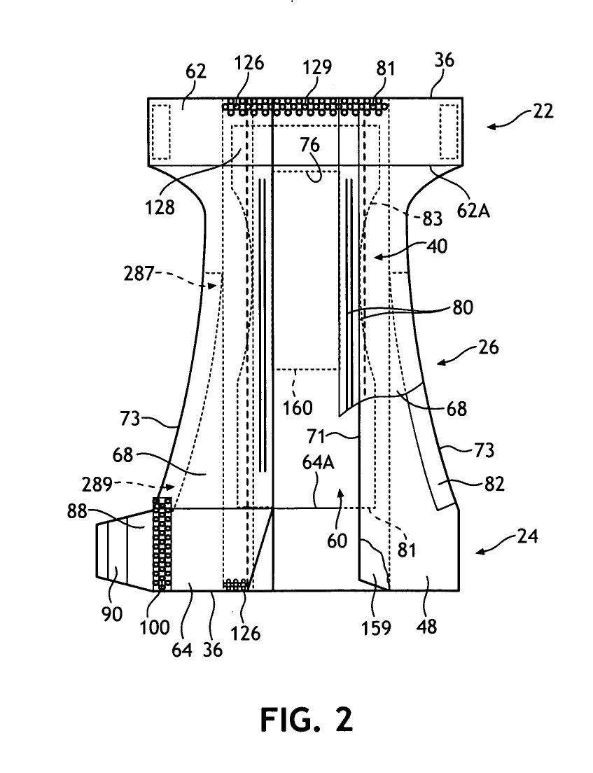

With reference now to the drawings and in particular to FIGS. 1 and 2, a disposable garment according to one embodiment of the present invention is described herein with reference to a disposable absorbent garment, and more particularly to a diaper, generally indicated at 20, intended to be worn about the waist and lower torso of an infant or toddler. It is understood, however, that the various aspects of the present invention are equally adaptable to other types of disposable garments such as adult incontinence garments, training pants, disposable swim pants and feminine hygiene garments. Disposable garments as referenced herein are intended for limited periods of use and are otherwise not intended for laundering. A disposable diaper, for example, is discarded after soiling by the wearer. Optionally, a disposable garment may include a replaceable absorbent insert wherein the remaining components of the garment may be reused several times before discarding.

The disposable diaper 20 of the illustrated embodiment generally has a front waist region 22, a back waist region 24, and a center region, otherwise referred to herein as a crotch region 26, extending longitudinally between and interconnecting the front and back waist regions. The front and back waist regions 22, 24 comprise those portions of the diaper 20 which, when worn, wholly or partially cover or encircle the waist and/or mid-lower torso of the wearer. The crotch region 26 generally is that portion of the diaper 20 which, when worn, is positioned between the legs of the wearer and covers the lower torso and crotch of the wearer.

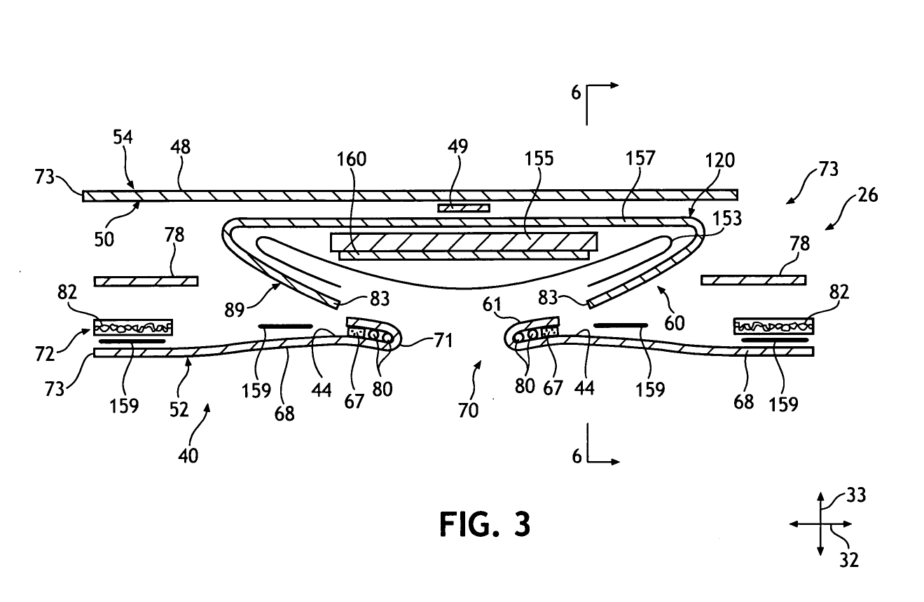

The diaper 20 has a longitudinal direction 30 and a lateral direction 32 thereof perpendicular to the longitudinal direction as indicated by the directional arrows provided in FIG. 1. The diaper also has a “z-direction” 33 (FIG. 3) which is generally normal to the longitudinal direction 30 and lateral direction 32. The various components of the diaper 20 described herein suitably define an inner surface 52 (FIG. 1) (otherwise referred to herein as a body-facing surface) which faces the wearer of the diaper and an outer surface 54 (FIG. 3) (otherwise referred to herein as a garment-facing surface) which faces away from the wear.

With further reference to FIG. 1, the diaper 20 also has a pair of laterally opposite side edges 34, and a pair of longitudinally opposite ends 36. The diaper 20 is illustrated in FIGS. 1 and 2 in an unfolded and laid flat (e.g., uncontracted) condition, similar to the configuration of the diaper prior to it being placed on the wearer. As worn, the diaper takes on a three-dimensional configuration (not shown) in which the side edges 34 of the diaper suitably define leg openings of the diaper 20 and the ends 36 together define a waist opening of the diaper 20.

The diaper 20 may be of various suitable shapes when unfolded and laid flat. For example, the diaper 20 may have an overall T-shape, hour-glass shape or a general I-shape as in the illustrated embodiment. Examples of diaper configurations suitable for use in connection with the instant application are described in U.S. Pat. No. 4,798,603 issued Jan. 17, 1989 to Meyer et al.; U.S. Pat. No. 5,176,668 issued Jan. 5, 1993, to Bernardin; U.S. Pat. No. 5,176,672 issued Jan. 5, 1993 to Bruemmer et al.; U.S. Pat. No. 5,192,606 issued Mar. 9, 1993 to Proxmire et al.; and U.S. Pat. No. 5,509,915 issued Apr. 23, 1996 to Hanson et al.; U.S. Ser. No. 11/215,978 filed Aug. 31, 2005 by VanGompel et al.; U.S. Ser. No. 11/118,619 filed Apr. 29, 2005 to Mullen et al.; and U.S. Ser. No. 10/735,978 filed Dec. 15, 2003 to Franke et al.; the disclosures of which are hereby incorporated by reference.

The illustrated diaper 20 (FIG. 3) suitably comprises an inner layer, generally indicated at 40, an outer layer, generally indicated at 48 in generally opposed (and more suitably superposed) relationship with the inner layer, and an absorbent assembly, generally indicated at 60, disposed between the inner and outer layers of the diaper for taking in and retaining body exudates released by the wearer. The inner layer 40 has an inner, or body-facing surface that at least in part defines the inner surface 52 of the diaper 20 and an outer, or garment-facing surface 44 (FIG. 3). The outer layer 48 (also commonly referred to as an outer cover) has an inner, or body-facing surface 50 (FIGS. 2 and 3) and an outer or garment-facing surface that defines the outer surface 54 of the diaper 20.

The inner layer 40 of the illustrated embodiment of FIG. 1 is suitably of a multiple component construction, and in particular comprises a front waist panel 62 (broadly, a first end panel of the inner layer) at least in part defining the front waist region 22 of the diaper 20 and a back waist panel 64 (broadly a second end panel of the inner layer) at least in part defining the rear waist region 24 of the diaper. The front and back waist panels 62, 64 are suitably longitudinally spaced from each other. For example, in the illustrated embodiment of FIG. 2 the front waist panel has a longitudinally outer end coterminous with the outer layer 48 to define a longitudinal end 36 of the diaper 20, and a longitudinally inner end 62A. The back waist panel 64 has a longitudinally outer end coterminous with the opposite end of the outer layer to define the opposite longitudinal end 36 of the diaper 20, and a longitudinally inner end 64A spaced longitudinally from the inner end 62A of the front waist panel so that the spacing therebetween at least in part defines the crotch region 26 of the diaper.

Referring to FIG. 2, the inner layer 40 further comprises a pair of laterally spaced side panels 68 that extend longitudinally through the crotch region 26 of the diaper 20, and more suitably extend from the front waist region 22 through the crotch region to the back waist region 24 of the diaper, and ever more suitably from one end 36 of the diaper 20 to the other end. Thus, it will be seen that the front and back waist panels 62, 64 and laterally spaced side panels 68 together define a central opening 76 of the inner layer 40 of the diaper 20 through which liquid, semi-liquid and solid exudates released by the wearer pass to the absorbent assembly 60. Each of the side panels 68 has a laterally inboard edge 71 and a laterally outboard edge 73. In the illustrated embodiment, the side edges of the front and back waist panels 62, 64 and the side edges of the outer layer 40 are coterminous and together define the lateral side edges 34 of the diaper 20 while the longitudinally inner ends 62A, 64A of the front and back waist panels 62, 64 and laterally inboard edges 71 of the side panels 68 together define the central opening 76 of the inner layer 40 of the diaper.

The side panels 68 are formed separate from the front and back waist panels 62, 64 and absorbent assembly 60 for subsequent assembly therewith. The side panels 68 are suitably constructed to be generally compliant, soft feeling, and nonirritating to the wearer's skin. In one suitable embodiment, each side panel 68 is stretchable (e.g., at least extensible), and is more suitably elastic, in the longitudinal direction 30 of the diaper 20.

As used herein, the term “stretchable” refers to a material that may be extensible or elastomeric. That is, the material may be extended, deformed or the like, without breaking, and may or may not significantly retract after removal of an extending force. The terms “elastomeric” or “elastic” are used interchangeably herein and refer to that property of a material where upon removal of an elongating force, the material is capable of recovering to substantially its unstretched size and shape or the material exhibits a significant retractive force. The term “extensible” refers to that property of a material where upon removal of an elongating force, the material experiences a substantially permanent deformation or the material does not exhibit a significant retractive force. In particular, elastomeric materials utilized in connection with the present invention may be elongated/extended or stretched in at least one direction without breaking by at least 25% (to at least 125% of its initial unstretched length) in at least one direction, suitably by at least 50% (to at least 150% of its initial unstretched length) and which will recover, upon release of the applied stretching or biasing force, at least 10% of their elongation. It is generally suitable that the elastomeric material or composite be capable of being elongated by at least 100%, more suitably by at least 200%, of its relaxed length and recover at least 30% and more suitably 50% of its elongation upon release of a stretching, biasing force, within about one minute.

Similarly, extensible or elongatable materials of the present invention may be capable of stretching in at least one direction without breaking by at least 25% (to at least 125% of its initial unstretched length) in at least one direction, suitably by at least 50% (to at least 150% of its initial unstretched length), more suitably by at least 100% (to at least 200% of its initial unstretched length). As an example, an extensible material having an initial unstretched length of 3 inches (7.6 centimeters) may be stretched without breaking to a stretched length of at least 3.75 inches (9.5 centimeters) in at least one direction (for the “by at least 25%” value).

In one suitable embodiment, illustrated in FIG. 1 each side panel 68 extends longitudinally from one longitudinal end 36 of the diaper 20 (i.e., the outer end of the front waist panel) to the opposite longitudinal end of the diaper (i.e., the outer end of the back waist panel). Accordingly, longitudinal end margins 75 of the side panels 68 generally underlie the front and back waist panels 62, 64 in the front and back waist regions 22, 24 of the diaper 20. The side panels 68 are suitably secured to the front and back waist panels 62, 64 along all or part of the end margins 75 of the side panels. More suitably, the side panels 68 are secured to the front and back waist panels 62, 64 from the laterally inboard edge 71, along each side panel end margin 75 that underlies the front and back waist panels, and to the laterally outward edge 73 of the outer cover in the region of end 36. In another embodiment (not shown) the side panels 68 may be secured to the front and back waist panels 62, 64 only across the longitudinally outer ends of the front and back waist panels corresponding to the longitudinal ends 36 of the diaper 20.

It is understood, however, that the side panels 68 need not extend to the longitudinal ends 36 of the diaper 20 to remain within the scope of this invention. For example, the side panels 68 may only be sized in length to underlie a portion of each of the front and back waist panels but otherwise terminate longitudinally inward of the ends 36 of the diaper 20. It is also contemplated that the side panels 68 may instead be sized in length to extend longitudinally into abutting (e.g., end-to-end) relationship with the longitudinally inner ends 62A, 64A of the front and back waist panels 62, 64 and be secured thereto without departing from the scope of this invention.

In another suitable embodiment, each side panel 68 is additionally stretchable (e.g., at least extensible), and may even be elastic, in the lateral direction 32 of the diaper 20. Providing lateral direction 32 stretchability to the side panel reduces the limiting effect that securing the side panel edge margins 75 to the front and back waist panels 62, 64 may have on the lateral stretch properties of the front and back waist panels. The lateral direction stretch capabilities of the side panel 68 also permit the side panel to stretch laterally during use at least along the longitudinal segment of the side panel extending between the longitudinally inner ends 62A, 64A of the front and back waist panels 62, 64, e.g., independent of the absorbent assembly, the outer layer and the front and back waist panels.

In the illustrated embodiment, each side panel 68 suitably comprises an elongate strip, such as a woven material, non-woven material, film or laminate comprised of one or more layers of such material. As an example, the side panels 68 may be suitably constructed of any of the materials from which a topsheet 153 or backsheet 157 of the absorbent assembly 60 may be constructed as described later herein. The side panels 68 are suitably constructed to be vapor permeable and liquid impermeable. However, the side panels 68 may alternatively be vapor and liquid impermeable, or vapor and liquid permeable, within the scope of this invention.

Each of the side panels 68 is suitably rendered elastic (e.g. elastomerically stretchable) in the longitudinal direction by securing one or more longitudinally extending elastic members along all or part of the length of the side panels. As an example, in the illustrated embodiment of FIG. 3, the elastic members in part comprise elastic strands 80 such as are known for use with containment flaps and waist elastics of conventional disposable garments such as diapers. In particular, one elastic member comprises a pair of elastic strands 80 extending longitudinally adjacent the laterally inboard edge 71 of the side panel 68. One or more elastic strands may be used. In addition, it is contemplated that one or more elastic bands (of greater width in the lateral direction 32) than thickness (in the z-direction) could be used instead of the substantially cylindrical-shaped bands shown in FIG. 3. The strip of material to which the elastic strands 80 are secured is folded over at the laterally inboard edge 71 of the side panel 68 to enclose the elastic strands therein. The elastic strands 80 suitably extend at least along the segment of the side panel 68 between the longitudinally inner ends 62A, 64A of the front and back waist panels 62, 64, but may extend along a greater or lesser extent of the length of the side panel. The elastic strands 80 (broadly, the elastic member) are suitably secured to the strip of material while the elastic strands are in a stretched condition.

Another elastic member of the side panel 68 comprises a single elastomeric band 82 extending longitudinally of the side panel in laterally spaced relationship with the elastic strands 80, and more suitably adjacent the laterally outboard edge 73 of the side panel 68. As illustrated in FIG. 3, the elastomeric band 82 is secured to the garment-facing side of the side panels 68 such that upon securement of the outboard edge 73 of the side panel 68 to the outer layer the band is coterminous. The elastomeric band 82 suitably extends longitudinally at least along the segment of the side panel 68 between the longitudinally inner end 64A of the back waist panel 64 and terminates in the crotch region 26. The elastomeric band 82 (broadly, the elastic member adjacent the outboard edge 73 of the side panel 68) is suitably secured to the side panel 68 while the elastomeric band, is in a stretched condition. In the alternative, elastomeric band 82 extends beyond the crotch region 26 and into the front waist region 22.

With reference to FIG. 1, the diaper 20 may further comprise a pair of ears 88 that may be fastened to the opposite waist region as the diaper is placed about a wearer or prefastened as such. Suitably, the ears 88 are joined at the laterally opposite side edges of the chassis adjacent the back waist region 24. More suitably, each ear 88 has a longitudinal length equal the back elastic waist panel longitudinal length 84, and aligned and joined adhesively, ultrasonically, by thermal bond to opposite ends of the back elastic waist panel 64 at the outboard edge 73. Ears 88 may be any extensible or elastic non-woven material, or suitably, a nonextensible nonwoven. One suitable material is spunbond-meltblown-spunbond.

In one embodiment of the present invention, the leg elastics defined by the elastomeric band 82 are located within close vicinity of the rear waist panel, and in particular, where the ears 88 are attached to the rear waist panel at the outboard edge 73. Suitably, the bond area 86 used to attach ears 88 to back waist panel 64 is an ultrasonic or thermal bond that joins the ears 88 and rear waist panel 64 to the outer layer 48. Most suitably, the materials within the bond are rendered non-extensible. When the bond area is non-extensible, lateral outward forces applied to ear 88 are more effectively translated to not only the back waist panel 64, but to the back waist region 24. Further, a force applied to ear 88 in an outward lateral direction 32 and an outward longitudinal direction 30 will more effectively transmit force from ear 88 to the elastomeric band 82, providing that the elastomeric band 82 is in close vicinity to the bond area 86. This synergism between the ear 88, the back waist panel 64, the elastic band 82, and the pleated absorbent assembly 60, helps to conform the back waist region 24 about the wearer's hips as the wearer moves about. The synergistic effect is even more pronounced when the side panels 68 are constructed to also be stretchable in the lateral direction 32.

In one embodiment of the present invention, the leg elastic defined by elastomeric band 82 is in close vicinity to the bond area 86 when it has an end 289 that terminates within about 7 mm of the rear waist panel. In another embodiment of the present invention, the elastomeric band 82 is in close vicinity to the bond area 86 when it has an end 289 that terminates within about 4 mm of the rear waist panel. In yet another embodiment of the present invention, the elastomeric band 82 is in close vicinity to the bond area 86 when it has an end 289 that overlaps the rear waist panel. In a further embodiment of the present invention, the bond area 86 may extend beyond longitudinally beyond the edge of the back waist panel and overlap the end 289 of the elastomeric band 82.

In one embodiment of the invention, the elastomeric band 82 has a lateral width at end 289 that is wider than the opposite end 287. In another embodiment of the invention, the elastomeric band 82 is tapered such that the lateral width of the end 289 is wider than the opposite end thereof (see, FIG. 1). In yet another embodiment, the elastic band 82 has a uniform width along its longitudinal length (see, FIG. 2).

While the elastic members are illustrated in FIG. 3 as comprising one or more elastic strands 80, and a single elastic band 82, it is contemplated that the elastic members may be other than elastic bands, such as elastic strands or other suitable elastic members, without departing from the scope of this invention. It is further understood that the stretchability and elasticity of the elastic member adjacent the inboard edge 71 of the side panel 68 may be different from the stretchability and elasticity of the elastic member adjacent the outboard edge 73 of the side panel.

The front and back waist panels 62, 64 of the inner layer 40 are suitably compliant, soft feeling, and nonirritating to the wearer's skin. The waist panels 62, 64 may have any suitable shape, such as rectangular (e.g., the front and back waist panel of the illustrated embodiment), trapezoidal or otherwise non-rectangular depending on the desired overall shape of the diaper 20. One or both of the front and back waist panels 62, 64 may extend laterally outward beyond laterally outboard edges 73 of the connecting members 68 and remain within the scope of this invention. Most suitably, the side edges of the front and back waist panels 62, 64 may be coterminous with the laterally outboard edges 73 of the connecting members 68.

The front and back waist panels 62, 64 may be suitably constructed to be vapor and liquid permeable, vapor permeable but liquid impermeable, or vapor and liquid impermeable. For example, the front and back waist panels 62, 64 may be suitably constructed of any of the liquid permeable materials from which a topsheet 153 of the absorbent assembly 60 is constructed as described later herein.

In particularly suitable embodiments, the front and back waist panels 62, 64 are also suitably stretchable, and are more suitably elastic (i.e., elastomerically stretchable) in at least the lateral direction of the diaper 20 to provide a retractive force about the waist of the diaper wearer. It is contemplated that the front and back waist panels 62, 64 may also be stretchable, and may even be elastic, in the longitudinal direction of the diaper.

Various materials may be used to construct the stretchable front and back waist panels 62, 64. For example, in one suitable embodiment each of the panels 62, 64 are elastic and comprise a three ply laminate such as a Stretch Bonded Laminate (SBL) that consists of two nonwoven facings attached to an elastic inner layer while the inner layer is in a stretched condition. One such suitable SBL material is disclosed in U.S. Pat. No. 4,657,802 to Morman, incorporated by reference herein.

Another suitable embodiment of the elastic panels 62, 64 is a Necked Bonded Laminate (NBL) that is also a three-ply laminate but the elastic inner layer is not prestretched prior to attaching the two nonwoven facings. For the NBL, the nonwoven facings are necked stretched and the elastic inner layer is attached between the necked facings. Other suitable elastically stretchable NBL materials as are described in U.S. Pat. No. 5,226,992 issued on Jul. 13, 1993 to Morman, the disclosure of which is hereby incorporated by reference.

In other embodiments the panels 62, 64 may be formed from elastically stretchable film materials. Such films may be elastic in the lateral direction, the longitudinal direction or both. One suitable elastic film is a breathable elastic film as described in U.S. patent application Ser. No. 10/703,761 filed on Nov. 7, 2003 and titled “Microporous Breathable Elastic Films, Methods of Making Same, And Limited Use or Disposable Product Applications,” the disclosure of which is hereby incorporated by reference. Use of such breathable, elastic films may provide additional benefits for the skin health of the wearers of the garments of the invention.

Additional examples of suitable breathable elastic film laminates for use in constructing the front and back waist panels 62, 64 are described in Provisional U.S. Patent Application Ser. No. 60/518,100 filed on Nov. 7, 2003 and titled “Microporous Breathable Elastic Film Laminates, Methods of Making Same, and Limited Use or Disposable Product Applications,” the disclosure of which is hereby incorporated by reference. Other suitable elastic laminates are described in U.S. patent application Ser. No. 10/743,245 filed on Dec. 22, 2003 and titled “Extensible and Stretch Laminates and Method of Making Same,” the disclosure of which is hereby incorporated by reference.

Other suitable elastic nonwoven materials from which the front and back waist panels 62, 64 may be constructed include elastomeric materials that are treated using nonwoven manufacturing processes such as meltblowing. Suitable elastomers that may be formed into microfibers/nonwoven webs are described in U.S. Pat. No. 4,663,220 issued to Wisneski et al. on May 5, 1987 and titled “Polyolefin-Containing Extrudable Compositions and Methods for Their Formulation Into Elastomeric Products Including Microfibers,” the disclosure of which is hereby incorporated by reference. Meltblowing of KRATON copolymers (“KRATON” is a trade designation of the Shell Chemical Company) to form composite nonwoven elastic webs is described in U.S. Pat. No. 4,657,802 issued to Morman on Apr. 14, 1987 and titled “Composite Nonwoven Elastic Web,” the disclosure of which is hereby incorporated by reference.

With particular reference to FIGS. 2 and 3, the absorbent assembly 60 has longitudinally opposite ends 81 and laterally opposite side edges 83, and is disposed below the central opening 76 of the inner layer 40, e.g., in the crotch region 26 of the diaper 20, to receive body exudates that pass through the inner layer. The absorbent assembly 60 is suitably sized in length to be about equal to the overall length of the diaper 20. The absorbent assembly 60 is also suitably sized in width to be slightly narrower than the overall width of the diaper 20 at the crotch region 26, but to otherwise extend laterally at least up to and more suitably laterally outward of the laterally inboard edges 71 of the side panels 68 (e.g., sized as wide as and more suitably wider than the central opening of the inner layer.

The illustrated absorbent assembly 60 suitably comprises the topsheet 153, the backsheet 157 and an absorbent structure 155 disposed therebetween for taking in and retaining liquid body exudates (e.g. urine where the garment is the diaper 20). In particular, the topsheet 153 and backsheet 157 are suitably in opposed, generally coextensive relationship with each other so that they together define longitudinal ends 81 and lateral side edges 83 of the absorbent assembly. It is contemplated, however, that the topsheet 153 and backsheet 157 need not be coextensive, e.g., one may be longer and/or wider than the other to singly define the longitudinal ends 81 and/or lateral side edges 83 of the absorbent assembly 60 without departing from the scope of this invention.

The topsheet 153 of the absorbent assembly 60 suitably defines an inner or body-facing surface of the absorbent assembly that is compliant, soft feeling, and nonirritating to the wearer's skin since it is exposed to the wearer's skin through the central opening 76 of the inner layer 40. Further, the topsheet 153 may be less hydrophilic than the absorbent structure 155, and is sufficiently porous to be liquid permeable so that liquid body exudates can readily penetrate through the topsheet to the absorbent structure 155. The topsheet 153 may be suitably formed from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers.

In particular, various woven and nonwoven fabrics may be used for the topsheet 153. For example, the topsheet 153 may be formed of a meltblown or spunbond web of polyolefin fibers. The topsheet layer 153 may also be a bonded-carded web composed of natural and/or synthetic fibers. The topsheet 153 may be composed of a substantially hydrophobic material, and the hydrophobic material may, optionally, be treated with a surfactant or otherwise processed to impart a desired level of wettability and hydrophilicity. As one example, the topsheet layer 153 may suitably comprise a nonwoven, spunbond, polypropylene fabric composed of about 2.8-3.2 denier fibers formed into a web having a basis weight of about 22 grams per square meter and a density of about 0.06 grams per cubic centimeter. The web can be treated with a suitable surfactant, such as about 0.28% Triton X-102 surfactant, which may be applied by any conventional means such as spraying, printing, brush coating or the like.

The backsheet 157 is suitably constructed to be liquid impermeable and may or may not be vapor permeable. For example, the backsheet 157 may be formed from a thin plastic film or other flexible liquid-impermeable material. In a more particular example, the backsheet 157 may be formed from a polyethylene film having a thickness of from about 0.013 millimeter (0.5 mil) to about 0.051 millimeter (2.0 mils). The backsheet 157 may also be formed from a polyolefin film having a nonwoven web laminated to the exterior surface thereof, such as a spunbond web of polyolefin fibers. For example, a stretch-thinned polypropylene film having a thickness of about 0.015 millimeter (0.6 mil) may be thermally laminated to a spunbond web of polypropylene fibers having a fiber diameter of about 15 to 20 microns, with the nonwoven web having a basis weight of about 17 grams per square meter (0.5 ounce per square yard). The backsheet 157 may in some embodiments include bicomponent fibers such as polyethylene/polypropylene bicomponent fibers. The backsheet 157 may also include a vapor permeable non-woven facing layer laminated to a micro-porous film to impart “breathability” to the barrier layer. Suitable “breathable” barrier layer 157 materials are described in U.S. Pat. No. 5,695,868 issued Dec. 9, 1997 to McCormack et al. and U.S. Pat. No. 5,843,056 issued Dec. 1, 1998 to Good et al., the disclosures of which are incorporated by reference to the extent they are consistent herewith.

The backsheet 157 may also be constructed of any of the materials disclosed later herein from which the outer layer 48 of the diaper 20 may be constructed. While the backsheet 157 may even be constructed of the same material as the outer layer 48, it is contemplated that the backsheet 157 and outer layer 48 may be constructed of different materials and remain within the scope of this invention. In particular, for example, the backsheet 157 need not have a non-woven or otherwise soft-textured outer surface because it is substantially covered by the outer layer 48 of the diaper 20 and therefore unexposed exterior of the diaper. Other suitable backsheet 157 constructions are disclosed in U.S. Pat. No. 6,217,563 (Van Gompel et al.), the disclosure of which is incorporated by reference to the extent it is consistent herewith. The backsheet 157 may optionally be stretchable, and may further optionally be elastic.

The absorbent structure 155 of the absorbent assembly 60 may suitably comprise a matrix of hydrophilic fibers, such as a web of cellulosic fluff, and may optionally further comprise a high absorbency material commonly known as superabsorbent material. For example, the absorbent structure 155 may include a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. Alternatively, the absorbent structure 155 may include a laminate of fibrous webs and superabsorbent material or other suitable matrix for maintaining a superabsorbent material in a localized area. The size and the absorbent capacity of absorbent structure 155 should be compatible with the size of the intended wearer and the liquid loading imparted by the intended use of the garment. Further, the size and the absorbent capacity of the absorbent structure 155 can be varied to accommodate disposable absorbent garment wearers ranging from infants through adults.

The high-absorbency material may be selected from natural, synthetic, and modified natural polymers and materials. The high-absorbency materials may be inorganic materials, such as silica gels, or organic compounds, such as crosslinked polymers. The term “crosslinked” refers to methods for effectively rendering normally water-soluble materials substantially water insoluble but swellable. Such methods include, for example, physical entanglement, crystalline domains, covalent bonds, ionic complexes and associations, hydrophilic associations such as hydrogen bonding, and hydrophobic associations or Van der Waals forces. Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and mixtures and copolymers thereof. Further polymers suitable for use in the absorbent structure 155 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and the like. Mixtures of natural and wholly or partially synthetic absorbent polymers may also be useful. The high absorbency material may be in any of a wide variety of geometric forms. As a general rule, the high absorbency material is in the form of discrete particles. However, the high absorbency material may also be in the form of fibers, flakes, rods, spheres, needles, or the like.

In general, the high-absorbency material is suitably present in the absorbent structure 155 in an amount from about 5 to about 90 percent by weight, more suitably in an amount of at least about 30 percent by weight, and even more suitably in an amount of at least about 50 percent by weight based on a total weight of the absorbent structure 155. For example, in a particular aspect, the absorbent structure 155 may comprise a laminate which includes at least about 50 percent by weight and more suitably at least about 70 percent by weight of high-absorbency material overwrapped by a fibrous web or other suitable material for maintaining the high-absorbency material in a localized area. An example of high-absorbency material suitable for use in the present invention is DRYTECH 2035 polymer available from Dow Chemical, a business having offices in Midland, Mich. Other suitable superabsorbents may include FAVOR SXM 880 polymer obtained from Stockhausen, a business having offices in Greensboro, N.C.

The absorbent structure 155 may further comprise a wrapsheet (not shown) at least covering the body-facing surface of the fibrous matrix, and more suitably enclosing the matrix in the wrapsheet. Alternatively, the wrapsheet may cover both the body-facing surface and the garment-facing surface of the matrix but not the sides of the matrix. The wrapsheet is suitably comprised of an absorbent material, or at least a liquid permeable material. For example, a suitable wrapsheet may comprise a meltblown web composed of meltblown fibers, such as meltblown polypropylene fibers. Another example of a suitable wrapsheet may comprise a low porosity cellulosic web, such as a tissue web composed of an approximately 50/50 blend of hardwood/softwood fibers.

With reference to FIGS. 2 and 3, the absorbent assembly 60 may further include a surge management layer 160 positioned between the topsheet 153 and the absorbent structure 155 to quickly take in and efficiently distribute liquid exudates to the absorbent structure 155. Suitable constructions and arrangements of surge management layers are well known to those skilled in the art. The surge management layer 160 is sized narrower than the width of the absorbent structure 155 and is sized substantially shorter than the length of the absorbent structure. The surge layer 160 is suitably positioned longitudinally nearer to the front end of the absorbent assembly where initial insults occur.

As illustrated in FIG. 3, the topsheet 153 and backsheet 157 of the absorbent assembly 60 may extend laterally beyond the side edges of the absorbent structure 155 to the side edges 83 of the absorbent assembly 60. That is, the width of the absorbent structure is less than the width of the topsheet 153 and/or backsheet 157 so that the side edges 83 of the absorbent assembly 60 are disposed laterally outward of the side edges of the absorbent structure. In particular embodiments, the topsheet 153 and backsheet 157 are suitably secured to each other along the longitudinal ends 81 and lateral side edges 83 of the absorbent assembly 60 by adhesive bonding, ultrasonic bonding, thermal bonding or other suitable securement technique. The topsheet 153 may be secured directly to the backsheet 157 as in the illustrated embodiment or indirectly, such as by an intermediate component.

The laterally outward extensions of the topsheet 153 and/or the backsheet 157 are suitably C-folded as illustrated in FIG. 3 such that lateral edge margins 89 of the absorbent assembly 60 (e.g., a lateral region at and/or adjacent to the side edges 83 of the absorbent assembly), and more suitably the garment-facing surface thereof, are in opposed relationship with the inner layer 40 of the diaper 20 for attachment thereto as will be described later herein. In particular, the outer or garment-facing surface of the absorbent assembly 60 at the lateral edge margins 89 thereof (e.g., the outer surface of the backsheet 157) are in opposed relationship with and secured to the outer or garment-facing surface 44 of the inner layer 40 and more suitably the side panels 68 of the inner layer. It is contemplated that the lateral extensions of the topsheet 153 and/or the backsheet 157 may instead be Z-folded or folded in another suitable manner which allows subsequent unfolding, or expansion, of the lateral extensions during loading of the absorbent assembly 60. It is also contemplated that the lateral extensions of the topsheet 153 and/or backsheet 157 may be folded such that the inner or body-facing surface of the absorbent assembly at the lateral edge margins 89 thereof are in opposed relationship with and secured to the garment-facing surface 44 of the inner layer 40.

The lateral extensions of the topsheet layer 153 and the barrier layer 157 allow the absorbent assembly 60 to expand in the z-direction 33 (FIG. 3) upon loading of the absorbent structure 155. The lateral extensions also allow for lateral expansion of the absorbent assembly 60 upon lateral elongation of the inner layer opening 76, such as where the front and back waist panels 62, 64 are stretched laterally and thus move the side panels 68 further apart.

With particular reference to FIG. 3, the lateral edge margins 89 of the absorbent assembly 60 are suitably secured to the side panels 68 by adhesive 159. The adhesive 159 may be applied as a uniform continuous layer of adhesive, a patterned layer of adhesive, a sprayed pattern of adhesive, an array of separate lines, swirls or dots of adhesive, or any other pattern of adhesive. It is understood that the lateral edge margins 89 of the absorbent assembly 60 may alternatively, or additionally be secured to the side panels 68 by ultrasonic bonding, thermal bonding or other suitable securement techniques.

The absorbent assembly 60 is also suitably secured along its longitudinal ends to the respective front and back waist panels 62, 64. The securement or bonding of absorbent assembly 60 along its longitudinal ends to the respective front and back waist panels 62, 64 is achieved by a bond means. The “bond means” is a structure that carries out the function of permanently bonding together the absorbent assembly 60 to other portions of the garment such as the front and back waist panels 62, 64; the outer layer 48 and/or the flap portions of side panels 68. Regardless of the particular embodiment of the invention, the term “permanent” used in conjunction with “bond” is meant to define a securement of one or more materials and/or layers of like materials, which is not intended to become unsecured or separate during the donning and wearing of the garment.

Limited bond means is a structure that carries out the function of bonding together the flap portion of a side panel 68 to the front or back waist panels 62, 64, without also bonding the side panel to the topsheet 153.

One possible bond structure used for the bond means or limited bond means is a material-to-material bond where the materials are fused together via exposure to heat or activity that causes heating, e.g. ultrasonic waves or pressure. Another possible bond structure includes the use of external devices such as adhesives, stitches, staples, rivets, and the like. Material-to-material bonds may be combined or used in combination with external devices. For instance an adhesive may be used in combination with an ultrasonic or thermal bond.

The bond means or limited bond means, whether a material-to-material bond or one created by external devices, may include a bond pattern. For instance, material-to-material bonds or adhesive bonds may cover an entire bond zone 126 continuously, or non-continuously, such as with a random or nonrandom array of bond points, stripes, and/or swirls. More specifically, the adhesive 78 may be applied as a uniform continuous layer of adhesive, a patterned layer of adhesive, a sprayed pattern of adhesive, an array of separate lines, swirls or dots of adhesive, or any other pattern of adhesive. Likewise, the material-to-material bonds may be applied as a uniform continuous bond area, a patterned bond area, an array of separate lines, swirls or dots of bond area, or any other pattern of bond area.

The bond means may be limited to a first bond zone 126. The first bond zone 126 is an area wherein bonding occurs between each and every layer in the z-direction of the garment 20. Any stretchability that would occur within the first bond zone when the layers were in an unbonded state would be greatly reduced, possibly so that no extensibility would occur within the first bond zone 126. For example, in FIG. 1, the first bond zone 126 bonds together the elastic waist panels 62, 64; the outer layer 48; one of the flap portions 70; and one of the absorbent assembly lateral side folds or pleats 120. In one embodiment, the first bond zone is located adjacent the waist opening, and may coincide with the edge at end 36.

The limited bond may be limited to a second bond zone 128. The second bond zone 128 is an area where one of the elastic waist panels 62, 64; one of the flap portions 70, and one of the absorbent assembly lateral side folds or pleats 120 are bonded together without bonding any of these components to the rest of the absorbent assembly 60 or outer layer 48. Suitably, the second bond zone 128 is located directly adjacent the first bond zone 126, and aligned in the longitudinal direction 30 therewith.

Except for bond zone 126 and the bond region 129 that laterally separates bond zones 126; the absorbent assembly 60 is otherwise free from securement to the front and back waist panels 62, 64 laterally between the side panels 68 particularly along the longitudinally inner ends 62A, 64A of the waist panels. In this manner, the front and back waist panels 62, 64 and the absorbent assembly 60 together form longitudinally opposite pockets during wear (e.g., that open longitudinally inward of the diaper 20) for collecting liquid, semi-liquid and solid exudates.

As further discussed herein, the side panels 68 and the outer layer 48 are stretchable in the lateral direction so that the attachment of the side panels to the outer layer does not inhibit the stretchability of either the side panels or the outer layer. Further, the laterally inboard edge 71 of the side panels 68 is free from attachment to the outer layer 48 so that the inboard edge of the side panels is positionable relative to and independent of the outer layer.

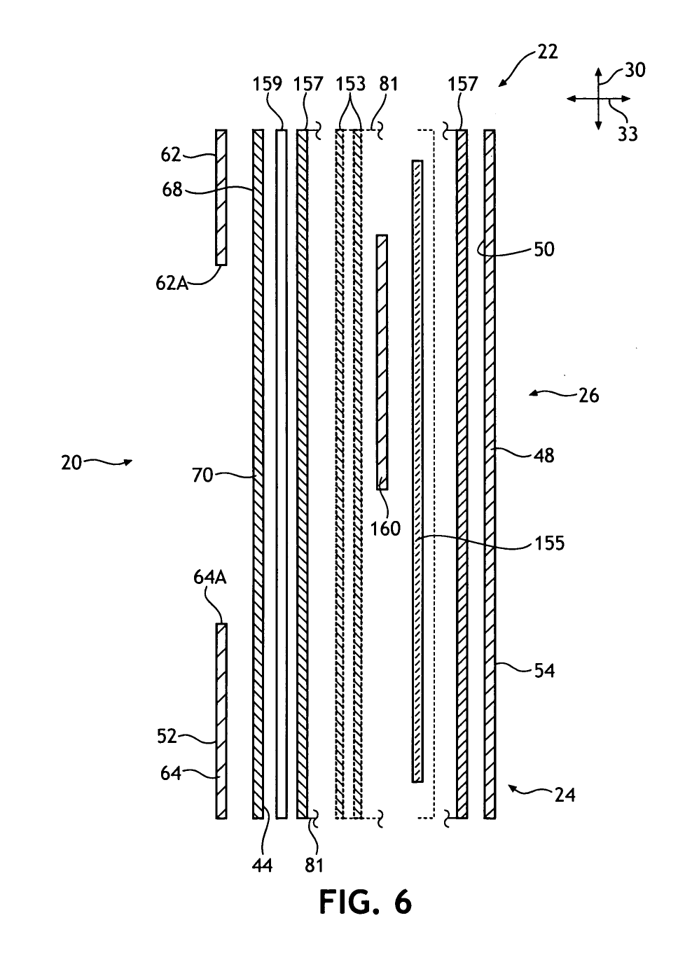

In a particularly suitable embodiment such as that illustrated in FIGS. 3 and 6, the lateral edge margins 89 of the absorbent assembly 60 are secured to the garment-facing surface of side panels 68 along a line of attachment, e.g., defined by the adhesive 159 in the illustrated embodiment, that extends generally longitudinally of the side panels along the entire length of the side panels. The line of attachment is suitably disposed laterally between the inboard and outboard edges 71, 73 of the side panel 68.

By keeping the first bond zone 126 relatively short in the longitudinal direction, the hip width 14a may increase to conform to the wearer's movements. For instance, a length of about 4 mm to about 25 mm may be desirable. In another embodiment, a length of about 4 mm to about 15 mm may be desirable. In yet another embodiment, a length of about 4 mm to about 10 mm may be desirable. The shorter the distance, the more easily the front waist region 22 and/or back waist region can expand and conform to a wearer's movements.

Because the inboard edges 71 of the side panels 68 are otherwise unsecured to the outer layer or other components of the diaper 20, the inboard edges of each side panel along such a segment are also positionable relative to and independent of the outer layer and the front and back waist panels. The outboard edges 73 of the side panels 68 are secured to the front and back waist panels 62, 64 at least along the segment of the side panels extending between the inner ends 62A, 62B of the waist panels. The outboard edges of each side panel along such a segment are also positionable relative to and independent of the front and back waist panels.

Thus, when a lateral force is applied to the elastic waist panels 62, 64, the back waist region 24 (adjacent the crotch region 26), as well as crotch region 26, is free to move laterally outward (outboard) in direction 32. To determine how far a pleated component may extend in the lateral direction 32, the following equation may be used. The final waist width 12 (Ww) is the maximum crotch width or hip width 14 (Hw) reduced by the number of folds or pleats times two, multiplied by the fold width.

Ww=Hw−(folds×2)×fold width

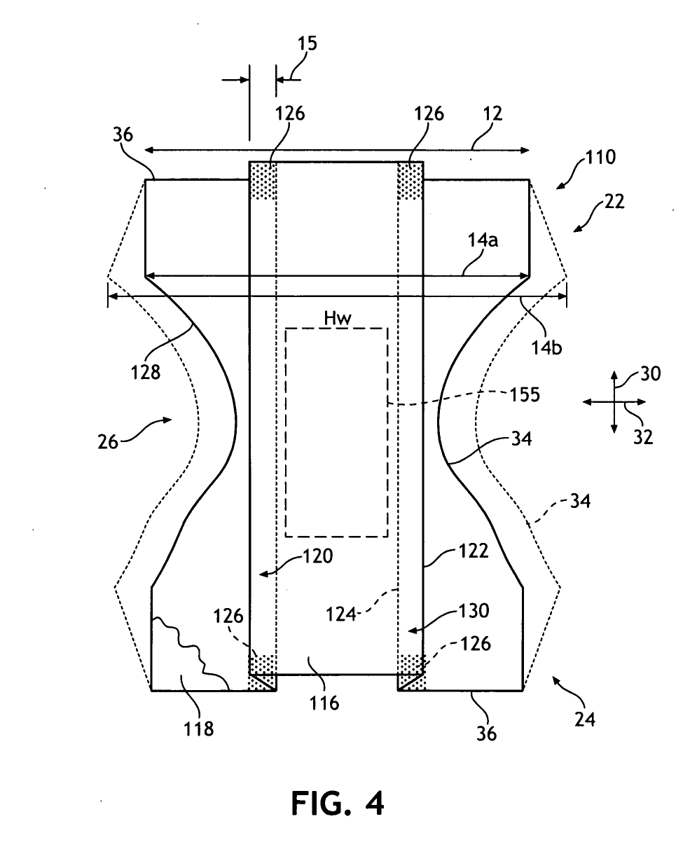

For example, a garment 20 that has a hip width (Hw) of 30 cm and two 3 cm folds at the waist region 22, has a front waist width 12 equaling 18 cm. (See FIG. 4 showing an embodiment of the invention demonstrating the waist and hip width.)

Referring to FIG. 3, the line of attachment between each side panel 68 and the edge margin 89 of the absorbent assembly 60 generally defines a laterally inner portion 70 or flap portion of the side panel between the line of attachment 159 and the inboard side edge 71 of the side panel, and a laterally outer portion 72 or leg cuff portion of the side panel between the line of attachment and the outboard side edge 73 of the side panel. In this manner, during wear the inner portion 70 of each side panel 68 broadly defines a containment flap along at least the longitudinal segment of the side panel between the longitudinally inner ends 62A, 64A of the front and back waist panels 62, 64. In particular, the containment flaps are oriented laterally inward and generally upright in the crotch region 26 to inhibit the lateral flow of body exudates out of the diaper 20. The outer portions 72 of the side panels 68 broadly define leg cuffs that are oriented laterally outward and generally downward to gasket about the wearer's legs to thereby reduce leakage and provide improved comfort and appearance.

Because the side panels 68 are generally hinged to the expandable absorbent assembly 60, upon wearing of the diaper 20 the containment flap portion (e.g., laterally inner portion 70) of the side panel is able to freely move relative to the absorbent assembly, the front and back waist panels 62, 64 and outer layer 48 up to its generally upright orientation at the crotch of the wearer while the leg cuff portion (e.g., laterally outer portion 72) of the side panel is able to freely move relative to the absorbent assembly and the front and back waist panels 62, 64 to a lower position nearer the thigh of the wearer.

In the illustrated embodiment of FIG. 3, the absorbent assembly 60 may be constructed to be generally free from securement to the outer layer 48 of the diaper 20. However, a more suitable embodiment shows a laterally central region of the absorbent assembly (e.g., laterally between the side edges of the absorbent structure 155) may be secured to the inner or body-facing surface 50 of the outer layer 48 by an adhesive 49 without departing from the scope of this invention (see FIG. 3).

The outer layer 48 is suitably extensible in at least the lateral direction in accordance with the lateral stretchability of the side panels 68, as shown in FIG. 3, in which the outer portion 72 of each side panel is secured to the outer layer. The outer layer 48 is also suitably soft-feeling, compliant and “breathable” or vapor permeable material. As an example, an extensible outer layer 48 can be composed of a necked fabric, a creped fabric, a micro-pleated fabric, polymer films or the like, as well as combinations thereof. The fabrics may be woven or nonwoven materials, such as spunbond fabrics. An example of a suitable extensible outer layer material is a 60% necked, polypropylene spunbond having a basis weight of about 1.2 osy.

For the purposes of the present disclosure, the term “% necked” or “percent neckdown” refers to a ratio or percentage determined by measuring the difference between the pre-necked dimension and the necked dimension of a neckable material, and then dividing that difference by the pre-necked dimension of the neckable material. The percentage of necking (percent neck) can be determined in accordance with the description in the above-mentioned U.S. Pat. No. 4,965,122 entitled REVERSIBLE NECKED MATERIAL AND PROCESS TO MAKE IT, by M. T. Morman which was issued Oct. 23, 1990 and is incorporated by reference herein.

Alternatively, the outer layer 48 of the diaper 20 may be suitably constructed to be liquid impermeable. For example, in one embodiment the outer layer 48 may be comprised of a thin plastic film or other flexible liquid-impermeable material. A particularly suitable outer layer 48 material is a polyethylene film having a thickness of from about 0.012 millimeter (0.5 mil) to about 0.051 millimeter (2.0 mils). To present such an outer layer 48 with a more clothlike feeling, the outer layer 48 may further comprise a polyolefin film having a nonwoven web laminated to the outer surface thereof, such as a spunbond web of polyolefin fibers. For example, a stretch-thinned polypropylene film having a thickness of about 0.015 millimeter (0.6 mils) may have thermally laminated thereto a spunbond web of polypropylene fibers having a thickness of about 1.5 to 2.5 denier per filament, with the nonwoven web having a basis weight of about 17 grams per square meter (0.5 ounce per square yard). Methods of forming such clothlike outer layers are known to those skilled in the art. Alternatively, or additionally, the outer layer 48 may be formed of a woven or nonwoven fibrous web that has been totally or partially constructed or treated to impart a desired level of liquid impermeability to selected regions that are adjacent or proximate to the absorbent assembly 60.

In other embodiments, the outer layer 48 may be suitably constructed to be permeable to water vapor and have a water vapor transmission rate (WVTR) of at least about 1000 g/m2/24 hours, more suitably at least about 1500 g/m2/24 hours, even more suitably at least about 2000 g/m2/24 hours, and still more at least about 3000 g/m2/24 hours. Materials which have a water vapor transmission rate less than those above do not allow a sufficient amount of air exchange and undesirably result in increased levels of skin hydration. A suitable technique for determining the WVTR value of a material is the test procedure standardized by INDA (Association of the Nonwoven Fabrics Industry), number IST-70.4-99, which is incorporated by reference herein. The testing device which may be used for WVTR measurement is known as the Permatran-W Model 100K manufactured by Mocon/Modern Controls, Inc., with an office in Minneapolis, Minn.

In another suitable embodiment, the outer layer 48 may comprise a microporous film/nonwoven laminate material comprising a spunbond nonwoven material laminated to a microporous film. For example, the laminate may include a 0.6 osy (20.4 gsm) polypropylene spunbond material thermally attached to an 18.7 gsm stretched microporous film. The film may include from about 20 percent to about 75 percent by weight calcium carbonate particulates and the remainder primarily low density polyethylene. The film is then stretched which causes the polyethylene component to stretch while the particulates remain unstretched, thus causing voids to develop around the calcium carbonate particles in the film. The resulting laminate may define a water vapor transmission rate of from about 1000 to about 5000 g/m2/24 hours.

The outer layer 48 of the diaper 20 is preferably formed from a stretchable material so as to be extensible or even elastic to facilitate stretching of the outer layer along with the inner layer 40 of the diaper 20 as well as the side panels 68 (where the side panels are secured to the outer layer such as in the embodiment of FIG. 3). When the outer layer 48 is formed of a stretchable material, the outer layer is suitably capable of stretching in at least the lateral direction 32 and may additionally be stretchable in the longitudinal direction 30. It is contemplated that where the outer layer 48 is stretchable in both directions, it may be extensible in both directions, elastic in both directions, or extensible in one direction and elastic in the other direction.

With further reference to FIG. 1, the diaper 20 may further comprise a pair of fastener tabs 90 employed on the ears 88 to secure the diaper about the waist of a wearer. Suitable fastener tabs 90 include hook-and-loop type fasteners, adhesive tape fasteners, buttons, pins, snaps, mushroom-and-loop fasteners, and the like. In the illustrated embodiment the fastener tabs 90 are secured between the outer layer 48 and the back waist panel 64 at the side edges of the diaper 20 by ultrasonic bonds or the like and extend laterally outward therefrom. Cooperating fasteners 92 may be provided on the outer layer 48 in the front waist region 22 of the diaper. Alternatively, the outer layer 48 may be constructed of a material to which the fastener tabs 90 may be suitably fastened.

With reference now to the drawings and in particular to FIG. 4, a disposable garment according to another embodiment of the present invention is described. In one embodiment of the present invention as shown in FIG. 4, there is a relatively simple disposable garment 110 for wearing about the lower torso of a person. As with the previous embodiment, the garment defines a longitudinal direction 30, a lateral direction 32, lateral side edges 34, front and back longitudinal ends 36; each of the ends having a waist width 14a between the lateral side edges 34. The garment has a front waist region 22, a back waist region 24, and a crotch region 26 extending longitudinally between and interconnecting the front waist region 22 and the back waist region 24. When the garment 110 is folded in about half at the crotch region 26, ends 36 may be joined to form a waist opening.

Disposable garment 110 may be constructed from either a single layer of material or multiple layers. In one embodiment, garment 110 is constructed from an outer layer 118 in opposed relationship with an inner layer 116. The inner and outer layers 116, 118 may be of the same size, or of different sizes. Further, the inner and outer layers 116, 118 may be aligned so that one or more of the ends 36 or side edges 34 are in alignment, as described in the previous embodiment. The inner and outer layers 116, 118 may be laminated together to form a single member, or remain separated over the majority of the surface area defined by the layers.

One or more pleats 120 may be formed in the garment by longitudinally folding the inner layer 116 and/or the outer layer 118 along the length of the garment 110 along fold lines 122 and 124. A single C-fold may be used as depicted; other fold configurations (e.g. Z-folds) may be made by adding additional fold lines adjacent the depicted fold lines 122 and 124. In addition, more pleats may be created across the waist width 12. For instance, there may be a total of three, four, five, six pleats, or more, as desired.

The waist width 12 is fixed by bonding the pleats 120 in at least a bond zone 126 adjacent the end 36. The purpose of bond zone 126 is to permanently join the inner and/or outer layers 116, 118 at the waist to form a permanent pleat so that a relative increase in hip width 14a can occur. This increase in width may occur regardless of whether the inner layer 116 and outer layer 118 are stretchable or elastic. However, the stretchability or elasticity of the materials may enhance the garment's ability to conform to a wearer. In addition, if only one of two or more layers includes one or more pleats 120, it is desirable that the non-pleated layer be at least stretchable so as not to prevent the pleated layer from expanding at the hip.

By keeping the bond zone 126 relatively short in the longitudinal direction, the hip width 14a may increase to conform to the wearer's movements. For instance, a length of about 4 mm to about 25 mm may be desirable. In another embodiment, a length of about 4 mm to about 15 mm may be desirable. In yet another embodiment, a length of about 4 mm to about 10 mm may be desirable. The shorter the distance, the more easily the front waist region 22 and/or back waist region can expand and conform to a wearer's movements.

Most desirably, a bond zone 126 acts to prohibit stretch in the area of the bond zone. For instance, if the inner and outer layers 16, 18 are stretchable or elastic, the waist width 12 will be prohibited from expanding in two ways: by the pleat structure, and by the lack of stretchability in the bond zone 126. By contrast, the garment is relatively uninhibited in its ability to expand provided that the bond zone is short enough so as not to prohibit the pleats 120 from unfolding across the hip width 14a. Thus, the hip width 14a may expand to hip width 14b when a lateral force is applied to the front or back waist region, as depicted in FIG. 4.

The embodiment of FIG. 4 may be enhanced by adding ears, fasteners, and absorbent assembly, or other features typical for a disposable absorbent product, as described herein for the previous embodiment. For example, an absorbent structure 155 may be placed between the inner and outer layers 116, 118.

When introducing elements of the present invention or the preferred aspect(s) thereof, the articles “a,” “an,” “the,” and “said” are intended to mean that there are one or more of the elements. The terms “comprising,” “including,” and “having,” are intended to be inclusive and mean that there may be additional elements other than the listed elements.

As various changes could be made in the above constructions without departing from the scope of the invention, it is intended that all matter contained in the above description or illustrated in the accompanying drawings shall be interpreted as illustrative and not in a limiting sense.

What is claimed is:

1. A disposable garment for wearing about the lower torso of a person, the garment having lateral side edges and front and back longitudinal ends defining a waist opening, the garment comprising:
an outer layer in opposed relationship with an inner layer;
wherein the inner layer has a pleat located at the waist opening, and
the pleat and the outer layer are joined together with a permanent bond.

2. The disposable garment according to claim 1 further comprising an absorbent member located between the outer layer and the inner layer.

3. A disposable garment having a waist opening and two leg openings for wearing about the lower torso of a person, the garment comprising:
a chassis comprised of an outer layer and an inner layer, the chassis defining a longitudinal direction, a lateral direction, lateral side edges, front and back longitudinal ends and together defining the waist opening; a front waist region and a rear waist region; and a crotch region extending longitudinally between and interconnecting the front waist region and the back waist region;
wherein the outer layer is in opposed relationship with the inner layer;
wherein the inner layer comprises:
an absorbent assembly having lateral side folds adapted to contact a body-facing surface of the absorbent assembly; and
a pair of side panels positioned at the lateral side edges of the chassis and that each define a flap portion;
wherein the absorbent assembly and each flap portion extend from the front longitudinal end to the back longitudinal end of the chassis; and
an elastic waist panel located at each of the front waist region and the rear waist region, each elastic waist panel having a waist-side edge and a crotch-side edge;
a bond means for permanently bonding together at a first bond zone: one of the elastic waist panels, the outer layer, one of the flap portions, and one of the absorbent assembly lateral side folds; wherein the first bond zone is located adjacent the waist opening; and
a limited bond means for permanently bonding together at a second bond zone, with the exclusion of the outer layer: one of the elastic waist panels, one of the flap portions, and one of the absorbent assembly lateral side folds;
wherein the second bond zone is located directly adjacent the first bond zone, and aligned in the longitudinal direction therewith.

4. The disposable absorbent garment according to claim 3 wherein the bond means comprises adhesive.

5. The disposable absorbent garment according to claim 3 wherein the bond means comprises a material-to-material bond.

6. The disposable absorbent garment according to claim 5 wherein the material-to-material bond comprises an array of bond points.

7. The disposable absorbent garment according to claim 5 wherein the material-to-material bond comprises an ultrasonic or thermal bond.

8. The disposable absorbent garment according to claim 3 wherein the limited bond comprises an adhesive bond between the side panel and the flap portions.

9. The disposable absorbent garment according to claim 3 wherein the absorbent assembly is comprised of an absorbent structure disposed between a topsheet and a backsheet.

10. The disposable absorbent garment according to claim 3 further comprising leg elastics positioned at each of the lateral side edges of the chassis, and extending from the rear waist region to the crotch region.

11. The disposable absorbent garment according to claim 3 further comprising leg elastics positioned at each of the laterally outboard edges of the chassis, wherein each leg elastic has a first end located within about 7 mm of the rear elastic waist panel, and a second end that is disposed in the crotch region.

12. The disposable absorbent garment according to claim 11 wherein each leg elastic further comprises a width, and wherein the width at the first end is greater than the width at the second end.

13. The disposable absorbent garment according to claim 12 wherein the leg elastic width is tapered.

14. The disposable absorbent garment according to claim 3 wherein the bond zone has a longitudinal length of about 4 mm to about 25 mm.

15. The disposable absorbent garment according to claim 3 wherein the bond zone has a longitudinal length of about 4 mm to about 10 mm.

16. The disposable absorbent garment according to claim 3 further comprising ear members joined at the lateral side edges of the chassis adjacent the rear waist region.

17. The disposable absorbent garment according to claim 3 further comprising ear members having a longitudinal length about equal to the rear elastic waist panel longitudinal length, and aligned and joined to opposite ends of the rear elastic waist panel.

18. A disposable absorbent garment for wearing about the lower torso of a person, the garment defining a waist opening, a front waist region and a back waist region, the garment comprising:
an outer layer in opposed relationship with an inner layer;
wherein the inner layer and the outer layer are pleated to form one or more pleats at the front waist region or the back waist region, and
wherein the one or more pleats comprises a permanent bond at a bond zone.

19. The disposable absorbent garment according to claim 18 wherein the bond zone is located adjacent the waist opening.

20. The disposable absorbent garment according to claim 18 wherein the inner layer and the outer layer is pleated together to form a common pleat.
